(19) United States
(12) Patent Application Publication
(10) Pub. No.: US 2021/0386814 A1
(43) Pub. Date: Dec. 16, 2021

(54) METHODS AND COMPOUND FOR THE TREATMENT OF MALARIA

(71) Applicants: Amanuel Tesfazion, Washington, DC (US); Lydia Seifu, Silver Spring, MD (US); Meried Bezuneh, Alexandria, VA (US)

(72) Inventors: Amanuel Tesfazion, Washington, DC (US); Lydia Seifu, Silver Spring, MD (US); Meried Bezuneh, Alexandria, VA (US)

(21) Appl. No.: 17/380,787

(22) Filed: Jul. 20, 2021

Related U.S. Application Data
(60) Provisional application No. 63/027,854, filed on May 20, 2020.

(51) Int. Cl. A61K 36/81; A61K 36/28; A61K 36/185; A01N 25/34; A01N 65/12; A01N 65/08; A61P 33/06; A01P 17/00

(52) U.S. Cl. CPC A61K 36/81; A61K 36/28; A61K 36/185; A01N 25/34; A61K 2236/333; A01N 65/08; A61P 33/06; A01P 17/00; A01N 65/12

(57) ABSTRACT

A method for the treatment of malaria disease, including administering to a subject in need thereof of an anti-pathogenic compound, such that the anti-pathogenic compound is derived from an herbal extract.

### CROSS REFERENCE TO RELATED APPLICATION

This application claims the benefit of, and incorporates by reference, U.S. provisional patent application Ser. No. 63/027,854, entitled “Methods and Compound for Treatment of Malaria,” which was filed on May 20, 2020.

### BACKGROUND

### 1. Field

The present general inventive concept relates generally to treatment of a parasitic disease, and particularly, to a plurality of methods and a compound for the treatment of malaria.

### 2. Description of the Related Art

Each year, 300-500 million people contract malaria and about 3 million die, most of which are children under five years old. In absolute numbers, malaria kills three thousand children per day under the age of five. Sadly, children under age five account for over sixty percent of total deaths. In 2018 alone, the number of deaths for children under five was approximately two hundred seventy-two thousand. The total number of deaths readily exceeds that from AIDS. Malaria is easily the world's largest parasitic disease, killing more people than any other communicable disease except tuberculosis. Malaria is a major public health problem in more than 100 countries, inhabited by a total of some 2.4 billion people, or close to half of the world's population.

Malaria is the leading cause of outpatient consultation and health facility admission. About 75% of the geographic area of Ethiopia has significant malaria transmission risk (defined as areas <2,000 m). With about sixty-eight percent (fifty-seven million) of the country's total population living in this area. Ethiopia spends millions of dollars to contain the transmission in terms of health care cost, drugs, and mosquito nets. Since malaria affects the small farmers, the engine of Ethiopian economy, it is a major impediment to national development.

The malaria parasite exhibits a complex life cycle involving an insect vector (i.e. mosquito) and a vertebrate host (e.g., humans and/or animals). Dissecting the life cycle and its biochemical path way is the key strategy for target selection and drug development.

A protozoan parasite known as Plasmodium falciparum (P. falciparum) is transmitted to humans by the females of the Anopheles species of mosquito. However, other known protozoan parasites are also responsible for causing malaria, including P. malariae, P. ovale, P. vivax, and P. knowlesi. There are about four hundred sixty species of Anopheles mosquito, but only sixty-eight transmit malaria. Anopheles gambiae is one of the best malaria vectors since it is long-lived, prefers feeding on humans, and lives in areas near human habitation. A. gambiae is typically found in Africa.

FIG. 1A illustrates a life cycle of Plasmodium falciparum.

Prior to transmission, Plasmodium falciparum resides within the salivary gland of the mosquito. The parasite is in its sporozoite stage (i.e. a sporozoite is a cell that infects a host, which for Plasmodium are motile) at this point.

As the mosquito takes its blood meal, it injects a small amount of saliva into the skin wound. The saliva contains antihemostatic and anti-inflammatory enzymes that disrupt the clotting process and inhibit the pain reaction. Typically, each infected bite contains anywhere from five to two hundred sporozoites which proceed to infect the host, such as a human. Once in the human bloodstream, the sporozoites only circulate for a matter of minutes before infecting hepatocytes (i.e. liver cells).

Referring to FIG. 1A, after circulating in the bloodstream, the P. falciparum sporozoites enter hepatocytes. At this point, the parasite loses its apical complex (i.e. a group of cytoskeletal structures and associated membrane-bounded organelles found at the anterior end of adult obligate intracellular protozoan parasites) and surface coat, and transforms into a trophozoite (i.e. a growing stage in the life cycle of some sporozoan parasites, where they are absorbing nutrients from the host). Within the parasitophorous vacuole (i.e. a structure produced by some parasites in the cells of its host that allows the parasite to develop while protected from the phagolysomes of the host cell) of the hepatocyte, P. falciparum undergoes schizogonic development (i.e. asexual reproduction by multiple fission, found in some protozoa, especially parasitic sporozoans). In this stage, the nucleus divides multiple times with a concomitant increase in cell size, but without cell segmentation. This exoerythrocytic (i.e. occurring outside red blood cells) schizogony stage of P. falciparum has a minimum duration of roughly 5.5 days. After segmentation, the parasite cells are differentiated into merozoites.

After maturation, the merozoites are released from the hepatocytes and enter the erythrocytic (i.e. red blood cells) portion of their life-cycle. Note that these cells do not reinfect hepatocytes.

FIG. 1B illustrates another view of a life cycle of Plasmodium falciparum.

Merozoite

Referring to FIG. 1B, after release from the hepatocytes, the merozoites enter the bloodstream prior to infecting red blood cells. At this point, the merozoites are roughly 1.5 μm in length and 1 μm in diameter, and use the apicomplexan invasion organelles (e.g., apical complex, pellicle, and surface coat) to recognize and enter the host erythrocyte.

The parasite first binds to the erythrocyte in a random orientation. It then reorients such that the apical complex is in proximity to the erythrocyte membrane. A tight junction is formed between the parasite and erythrocyte. As it enters the red blood cell, the parasite forms a parasitophorous vesicle, to allow for its development inside the erythrocyte.

This tight entry junction is a potential drug target that could prevent the merozoite from entering erythrocyte.

During this stage, the merozoite rapidly reproduces asexually to create more merozoites, until the erythrocyte bursts and releases more merozoites, such that the merozoite cycle repeats.

Trophozoite

After invading the erythrocyte, the parasite loses its specific invasion organelles (e.g., apical complex and surface coat) and de-differentiates into a round trophozoite located within a parasitophorous vacuole in the red blood cell cytoplasm. The young trophozoite (or “ring” stage, because of its morphology on stained blood films) grows substantially before undergoing schizogonic division.

FIG. 10 illustrates a life cycle of Plasmodium vivax.

Schizont

Referring to FIG. 10, at the schizont stage, the parasite replicates its DNA multiple times without cellular segmentation. These schizonts then undergo cellular segmentation and differentiation to form roughly 16-18 merozoite cells in the erythrocyte. The merozoites burst from the red blood cell, and proceed to infect other erythrocytes. The parasite is in the bloodstream for roughly 60 seconds before it has entered another erythrocyte.

This infection cycle occurs in a highly synchronous fashion with roughly all the parasites throughout the blood in the same stage of development. This precise clocking mechanism has been shown to be dependent on the human host's own circadian rhythm. Specifically, as a result of the circadian rhythm, temperature changes within the human body seem to play a role in the development of P. falciparum within the erythrocytic stage.

FIG. 1D illustrates an erythrocytic stage of Plasmodium falciparum.

Referring to FIG. 1D, within the red blood cell, the parasite metabolism depends greatly on the digestion of hemoglobin.

Infected erythrocytes are often sequestered in various human tissues or organs, such as the heart, liver, and brain. This is caused by parasite-derived cell surface proteins being present on the red blood cell membrane, and it is these proteins that bind to receptors on human cells. Sequestration in the brain causes cerebral malaria, a very severe form of the disease, which increases the host's likelihood of death.

The parasite can also alter the morphology of the red blood cell, causing knobs on the erythrocyte membrane. Oxygen radicals and heme (i.e. an iron-containing compound of the porphyrin class which forms the nonprotein part of hemoglobin and some other biological molecules) are released during proteolysis (i.e. the breakdown of proteins or peptides into amino acids by the action of enzymes) and must be detoxified by dismutation (i.e. a disproportionation reaction, especially in a biological context, in which oxidized and reduced forms of a chemical species are produced simultaneously) and polymerization (i.e. a process of reacting monomer molecules together in a chemical reaction to form polymer chains or three-dimensional networks), respectively. Understanding the disposition of hemoglobin has allowed identification of essential processes and metabolic weak points that can be exploited to combat this scourge of mankind.

As the parasite matures, a large volume of hemoglobin is ingested by means of a cytosomal system (i.e. a cell mouth that is part of a cell specialized for phagocytosis, usually in the form of microtubule-supported funnel or groove, such that food is directed into the cytosine and sealed into vacuoles) that is formed by invagination of the parasitophorous vascular membrane and the parasite plasma membrane that separate parasite and erythrocyte cytoplasm. The protease enzyme the parasite uses to digest hemoglobin may be a target for control.

In humans, hemozoin (i.e. a residual malarial pigment) from lysed schizonts accumulates in the mononuclear phagocyte system (i.e. part of the immune system that consists of phagocytic cells located in reticular connective tissue. It consists primarily of monocytes and macrophages, and they accumulate in lymph nodes and the spleen. This system was previously known as the reticuloendothelial system), turning the liver and spleen black in cases of chronic or repeated infection. Heme polymerization may be an other potential drug target.

Hemozoin is formed from heme molecules that are liberated by degraded hemoglobin. A malarial parasite produces large quantities of hemozoine which occurs in the parasite food vacuole using aspartic protease (i.e. a catalytic type of protease enzyme that use an activated water molecule bound to one or more aspartate residues for catalysis of their peptide substrates) and cysteine protease (i.e. an enzyme that degrades proteins).

Therefore, malarial hemozoin plays an important role as a target for antimalarial drugs and in disease pathogensis. A new assay for hemozoin was developed in which the hemozoin was separated from cells by filtration. Hemozoin may also influence the mode of action of chloroquine and artimisinin. The synthesis of hemozoin is inhibited by chloroquine in cell free extracts and in both Plasmodium falsiparum and Plasmodium berghei.

FIG. 1E illustrates various stages of Plasmodium falciparum.

Ookinete

Referring to FIG. 1E, the diploid ookinete is an invasive form of P. falciparum within the mosquito. It traverses the peritrophic membrane (i.e. a semi-permeable, non-cellular structure which surrounds the food bolus in an organism's midgut) of the mosquito midgut and cross the midgut epithelium (i.e. the thin tissue forming the outer layer of a body's surface and lining the alimentary canal and other hollow structures). Once through the epithelium, the ookinete enters the basil lamina (i.e. a layer of extracellular matrix secreted by the epithelial cells, on which the epithelium sits) and forms an oocyst (i.e. a thick-walled structure within a mosquito's outer gut lining).

During the ookinete stage, genetic recombination can occur. This takes place if the ookinete was formed from male and female gametes derived from different populations. This can occur if the human host contained multiple populations of the parasite or if the mosquito fed from multiple infected individuals within a short time-frame.

Parasitophorous Vacuole

Within a red blood cell, P. falciparum resides inside the parasitophorous vacuole. This is formed during erythrocyte invasion. The proteins originating in the parasite pass through the membrane of the parasitophorous vacuole and are transported to the cytoplasm or membrane of the erythrocyte.

FIG. 1F illustrates a transmission electron micrograph of a Plasmodium falciparum trophozite inside an erythrocyte.

Referring to FIG. 1F, c. represents a Cystom, v. represents a transport vesicle, and dv. represents a digestive vacuole.

The strongest argument that hemoglobin degradation is necessary for Plasmodium survival comes from studies with protease inhibitors. When hemoglobin proteolysis is blocked, parasite development is interrupted. As such, the proteases of plasmodium may be a key molecular target for drug development. The antimalarial research in Ethiopia will investigate the molecular cross talks between the isolated lead molecules and the proteases of plasmodium in terms of inhibition, geometric hindrance, and molecular interactions.

Some drugs such as choroquine, mefloquine, pyrimeethamine, dapsone, and cycloguanil have been used for years against malaria. However, the increase of resistant malaria parasite strains represents the largest obstacle to antimalaria chemotherapy. The massive use of classical antimalarial drugs prompted fast evolution of drug resistant strains of P. falciparum, which requires urgent development. As malaria is a disease that most affects poor people from poor countries, the biggest pharmaceutical industries are generally not interested in spending efforts in research in this area.

For many years, major antimalarial drugs consisted of natural products, but since the 1930s, these drugs have been largely replaced with a series of synthetic drugs. For many years, quinine (i.e. a compound derived from the bark of cinchona tree) remained the major antimalarial drug, but from 1930s this natural product was largely replaced by a series of synthetic drugs including 8-aminoquinolines (e.g. primaquine), 4-aminoquinolines (e.g. chloroquine, amodiaquine) and folic acid synthesis inhibitors (e.g. proguanil, pyrimethamine). A large number of natural products have been shown to inhibit the growth of one or more species of protozoa, but very few are proven to be selectively toxic to the parasite. Moreover, the effectiveness of any chemotherapeutic agent is dependent upon a favorable therapeutic ratio. In other words, the drug must kill and/or inhibit the parasite, but have little to no toxicity to the host. (See https://www.ncbi.nlm.nih.gov/pmc/articles/PMC4075661/).

In more recent years, global progress in reducing malaria has decreased the incidence of cases from 2000 to 2015 by thirty-seven percent and the number of deaths has decreased by sixty percent. Despite the substantial progress, more efforts need to be done for treatment to further the decline of the prevalence of malaria.

Therefore, there is a need for an effective remedy and/or drug that is natural, inexpensive, and non-toxic, such that the drug is effective against sensitive and drug-resistant plasmodium strains. As such, there is a need for a method and compound for the effective treatment of malaria.

### SUMMARY

The present general inventive concept provides a plurality of methods and a compound for the treatment of malaria.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a method for the treatment of malaria disease, including administering to a subject in need thereof of an anti-pathogenic compound, such that the anti-pathogenic compound is derived from an herbal extract.

The herbal extract may be a glycol derivative.

The glycol derivative may be diethylene glycol dibenzoate.

The malaria may be caused by Plasmodium falciparum.

The anti-pathogenic compound may boost an immune system of the subject.

Boosting the immune system may include revitalizing at least one of a dysfunctional monocyte and a dysfunctional macrophage.

Boosting the immune system may include stimulating production of gamma interferon.

Boosting the immune system may use only the anti-pathogenic compound.

The foregoing and/or other features and utilities of the present general inventive concept may also be achieved by providing a method for the treatment of malaria disease, including administering to a subject in need thereof of an anti-pathogenic compound, such that the anti-pathogenic compound is derived from at least one Ethiopian plant.

The at least one Ethiopian plant may be at least one of Withania Somnifera leaf, Withania Somifera root, Eucelea root aqueous extract, Phytolacca dodecandra, Vernonia galamensis ethiopica, and Moringa.

The malaria may be caused by Plasmodium falciparum.

The foregoing and/or other features and utilities of the present general inventive concept may also be achieved by providing a method of preventing at least one mosquito from crossing a barrier, including applying a mosquito repellant insecticide to the barrier, such that the mosquito repellent insecticide is derived from at least one Ethiopian plant.

The at least one Ethiopian plant may be at least one of Vernonia galamensis ethiopica and Phytolacca dodecandra.

The barrier may be a bed net.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1A illustrates a life cycle of Plasmodium falciparum;

FIG. 1B illustrates another view of a life cycle of Plasmodium falciparum;

FIG. 10 illustrates a life cycle of Plasmodium vivax;

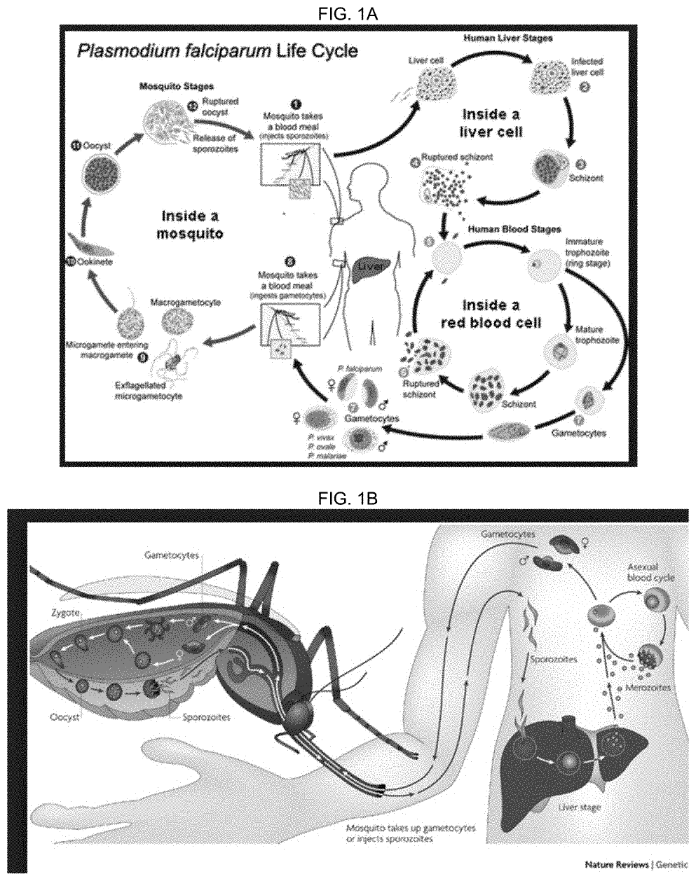

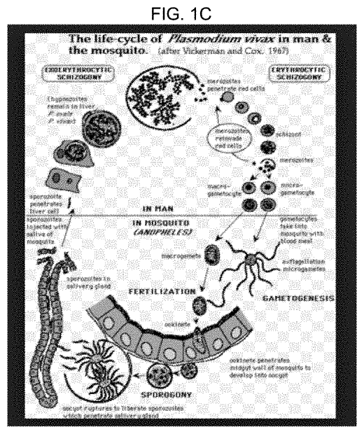

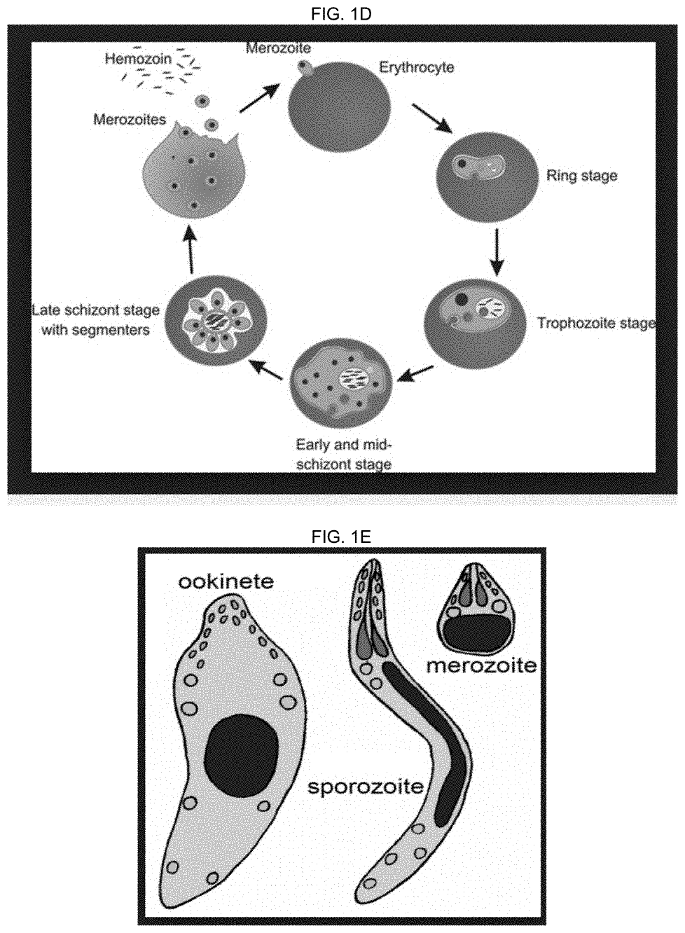

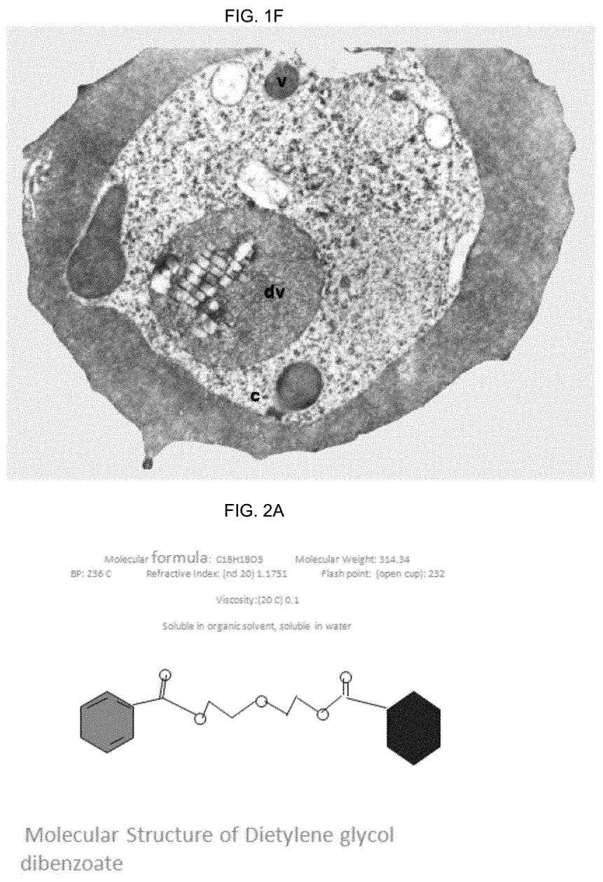

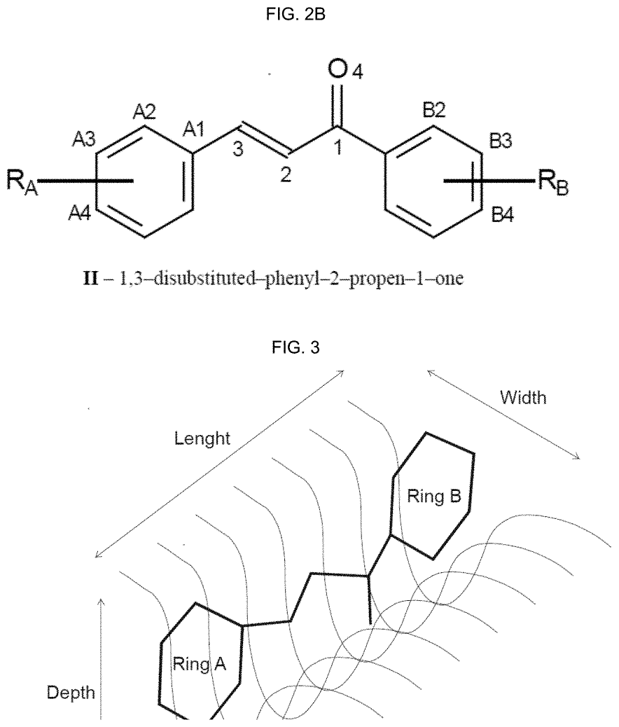

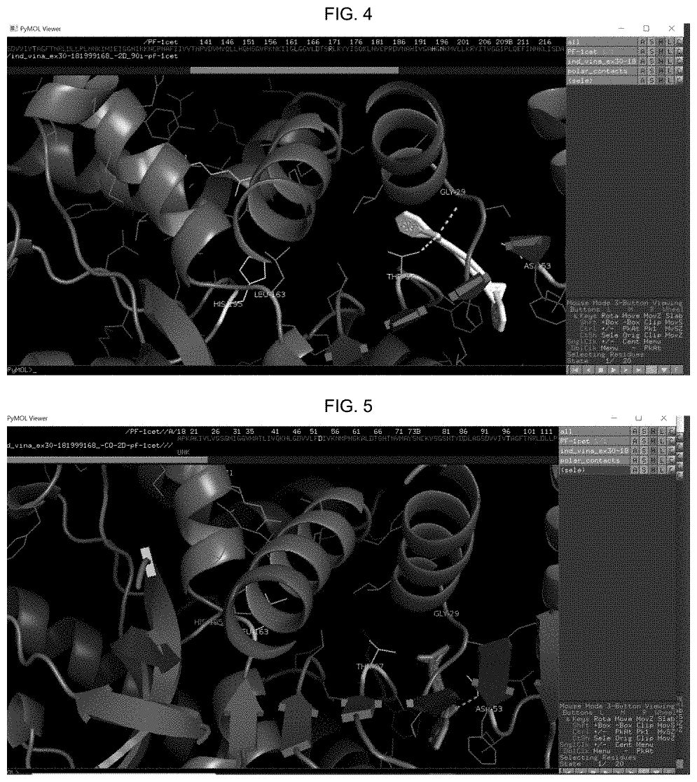

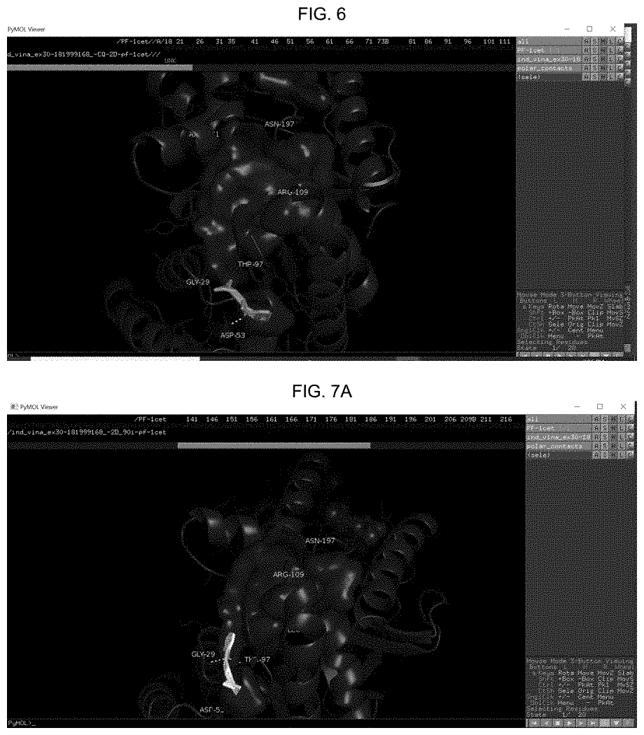

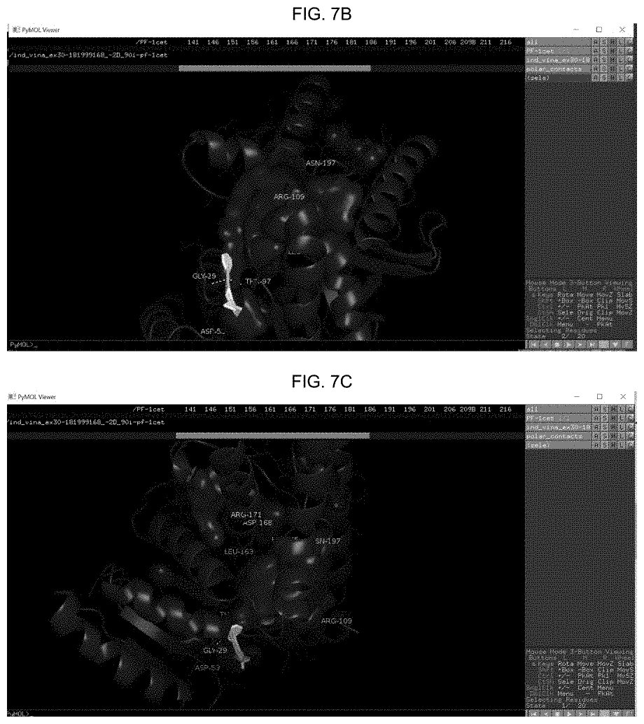

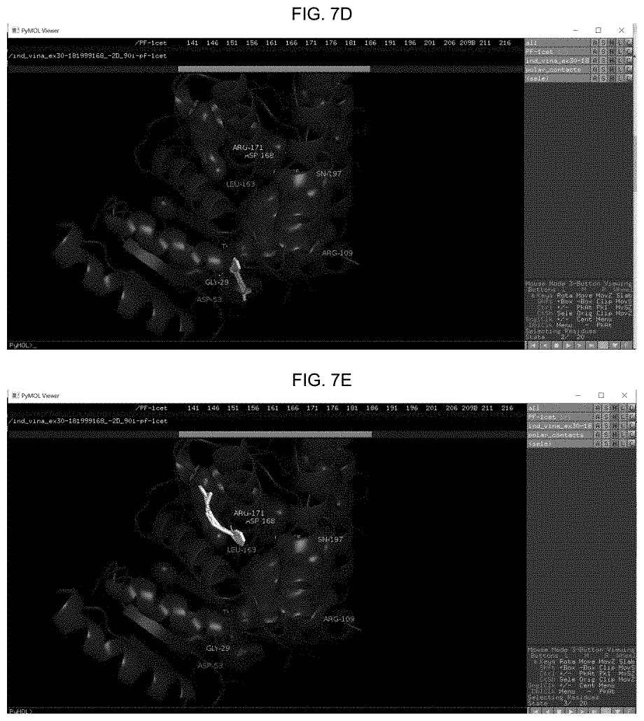

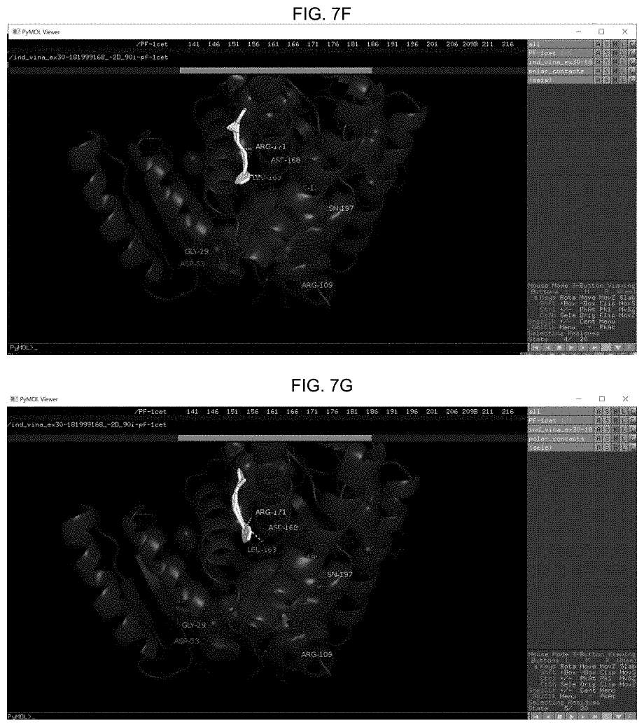

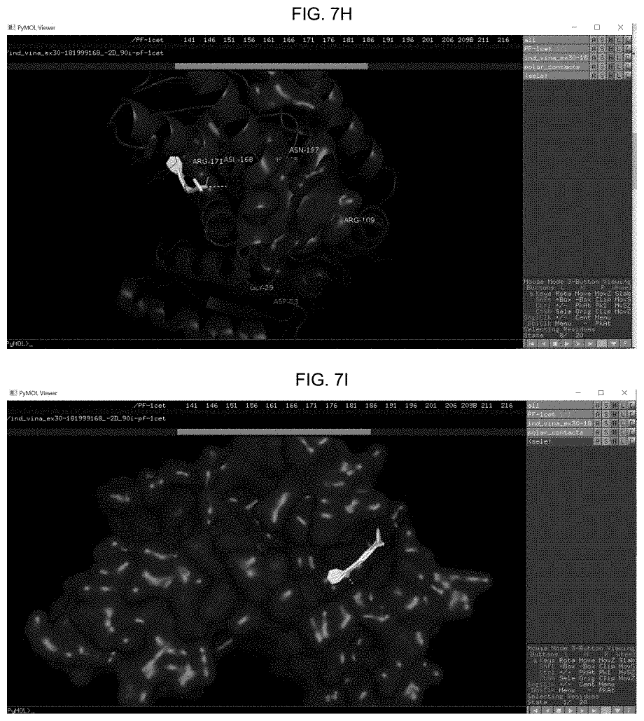

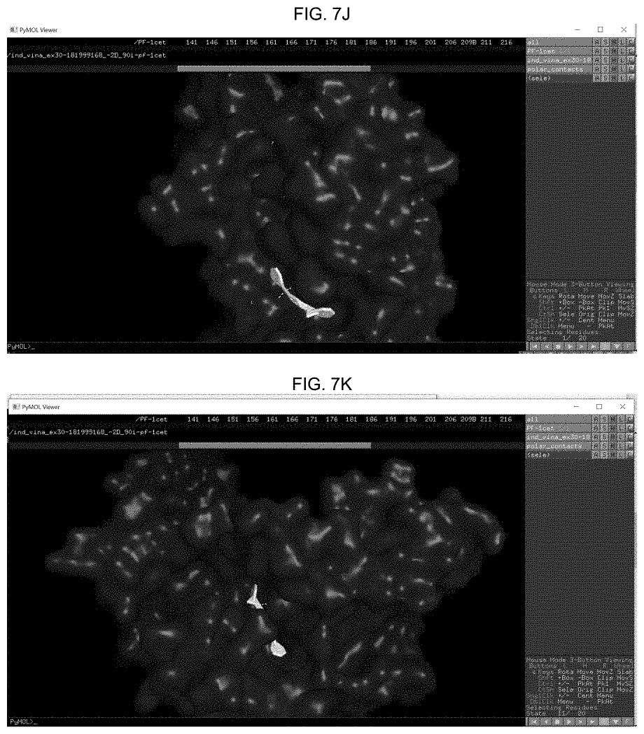

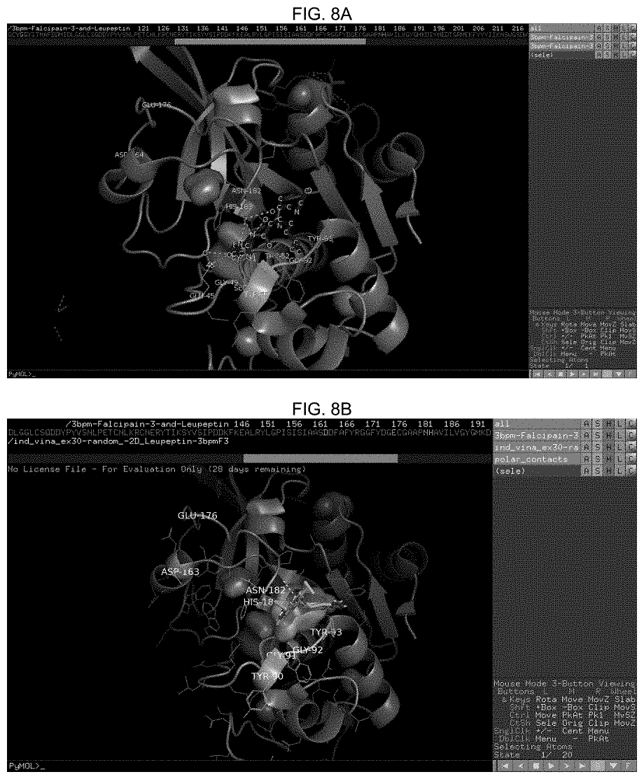

FIG. 1D illustrates an erythrocytic stage of Plasmodium falciparum;

FIG. 1E illustrates various stages of Plasmodium falciparum;

FIG. 1F illustrates a transmission electron micrograph of a Plasmodium falciparum trophozite inside an erythrocyte;

FIG. 2A illustrates a molecular structure of an anti-pathogenic compound, known as 90I (diethylene glycol dibenzoate), according to an exemplary embodiment of the present general inventive concept;

FIG. 2B illustrates a molecular structure of the anti-pathogenic compound, known as 90I, according to an exemplary embodiment of the present general inventive concept;

FIG. 3 illustrates a molecular structure of chloroquine;

FIG. 4 illustrates a first pose of 90I including two hydrogen bonds (H bonds), according to an exemplary embodiment of the present general inventive concept;

FIG. 5 illustrates a first pose of chloroquine including one H bond;

FIG. 6 illustrates the first pose of chloroquine disposed in a pocket;

FIG. 7A illustrates the first pose of 90I disposed in the pocket, according to an exemplary embodiment of the present general inventive concept;

FIG. 7B illustrates a second pose of 90I in the pocket, according to an exemplary embodiment of the present general inventive concept;

FIG. 7C illustrates another view of the first pose of 90I in the pocket, according to an exemplary embodiment of the present general inventive concept;

FIG. 7D illustrates another view of the second pose of 90I in the pocket, according to an exemplary embodiment of the present general inventive concept;

FIG. 7E illustrates a third pose of 90I in the pocket, according to an exemplary embodiment of the present general inventive concept;

FIG. 7F illustrates a fourth pose of 90I in the pocket, according to an exemplary embodiment of the present general inventive concept;

FIG. 7G illustrates a fifth pose of 90I in the pocket, according to an exemplary embodiment of the present general inventive concept;

FIG. 7H illustrates a sixth pose of 90I in the pocket, according to an exemplary embodiment of the present general inventive concept;

FIG. 7I illustrates a first surface view of the first pose of 90I in the pocket, according to an exemplary embodiment of the present general inventive concept;

FIG. 7J illustrates a second surface view at a different angle with respect to the first surface view of the first pose of 90I in the pocket including two hydrogen bonds in dotted yellow lines, according to an exemplary embodiment of the present general inventive concept;

FIG. 7K illustrates 90I disposed at a deeper portion of the pocket including the two hydrogen bonds in dotted yellow lines, according to an exemplary embodiment of the present general inventive concept;

FIG. 8A illustrates Leupeptin in blue and F3 P. Falciparum in gray from Protein Data Bank (PDB), including one section of F3 having residues as green dots;

FIG. 8B illustrates Leupeptin in green and F3 P. Falciparum in gray from PDB;

FIG. 8C illustrates a first pose of Leupeptin in green interacting with ASN 182 and F3 P. Falciparum in gray including residues in blue;

FIG. 9A illustrates the first pose of 90I using residues of falcipain-III protein 3BPM PDB, according to an exemplary embodiment of the present general inventive concept;

FIG. 9B illustrates the fifth pose of 90I on residues of falcipain-III protein 3BPM PDB, according to an exemplary embodiment of the present general inventive concept;

FIG. 9C illustrates the first pose of 90I on residues of falcipain-III protein 3BPM PDB, according to an exemplary embodiment of the present general inventive concept;

FIG. 9D illustrates a twelfth pose of 90I on Gly 49 residues of falcipain-III protein 3BPM PDB, according to an exemplary embodiment of the present general inventive concept;

FIG. 9E illustrates a fifteenth pose of 90I on GLN 45 and Gly 92 residues of falcipain-III protein 3BPM PDB, according to an exemplary embodiment of the present general inventive concept;

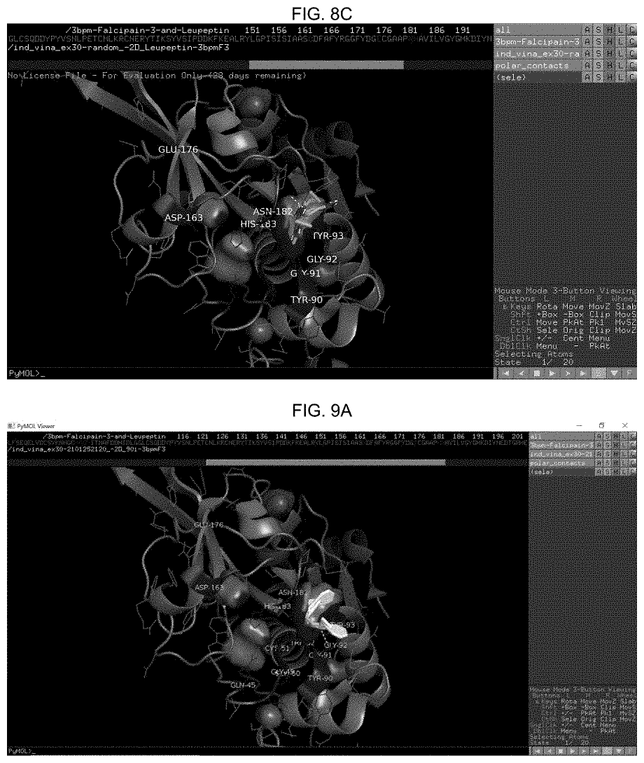

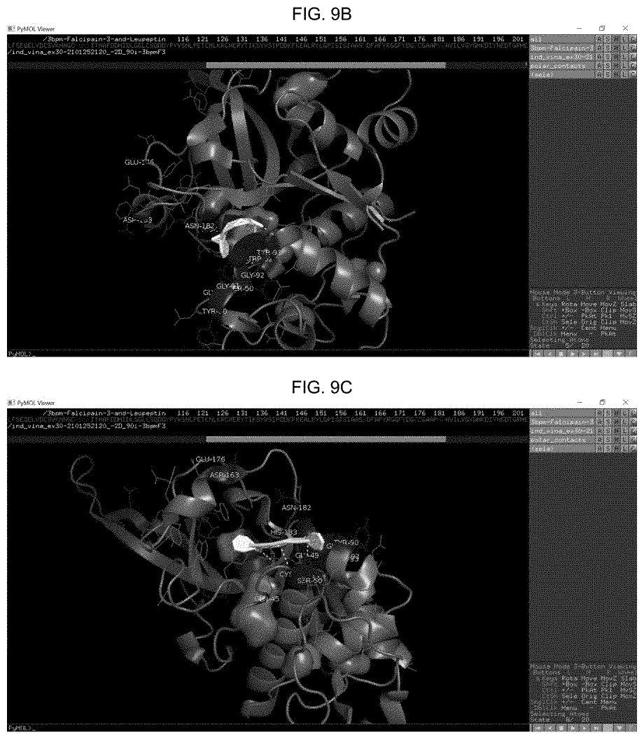

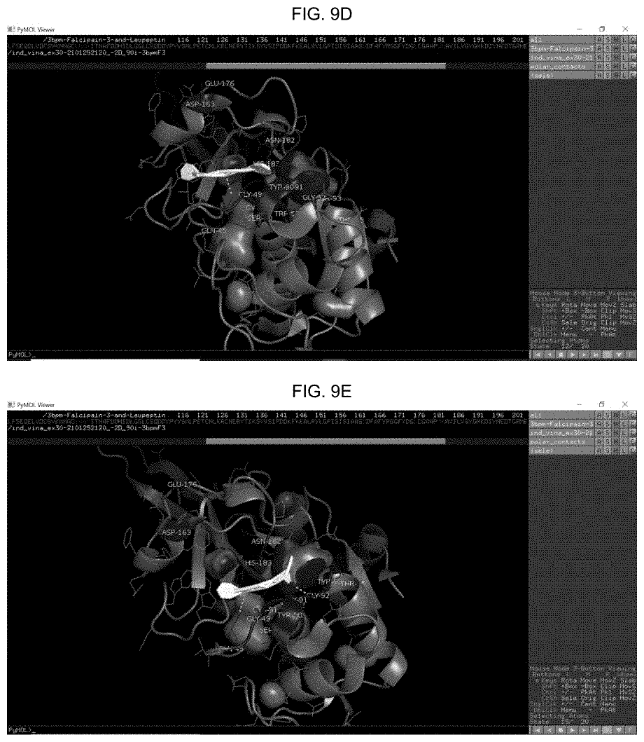

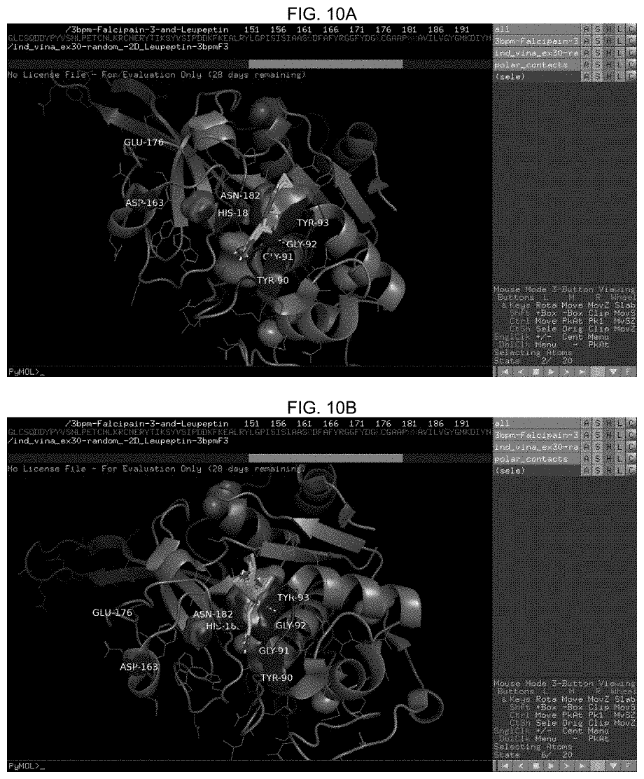

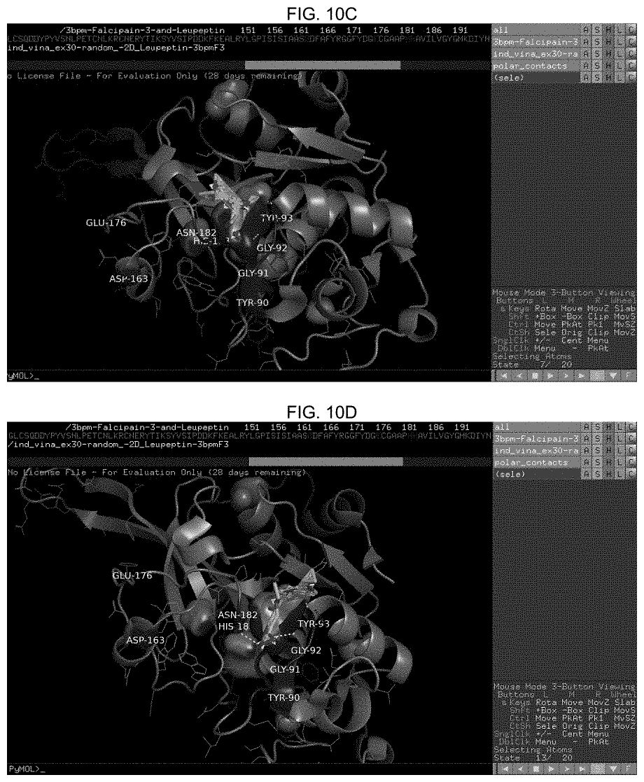

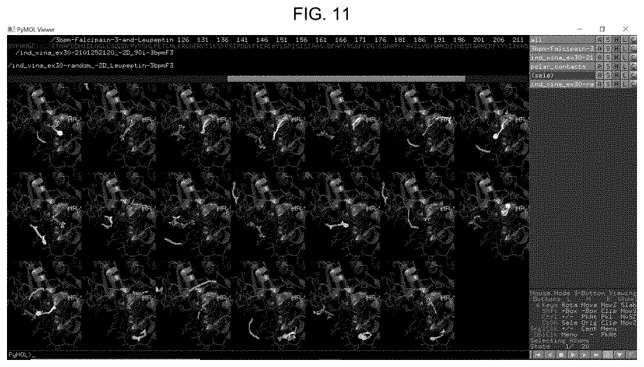

FIG. 10A illustrates a second pose of Leupeptin on GLY 92 residues with a hydrogen bond of falcipain-III protein 3BPM PDB;

FIG. 10B illustrates a sixth pose of Leupeptin on GLY 92 and TYR93 residues with the hydrogen bond of falcipain-III protein 3BPM PDB;

FIG. 100 illustrates a seventh pose of Leupeptin on GLY 92 and TYR93 residues with the hydrogen bond of falcipain-III protein 3BPM PDB;

FIG. 10D illustrates a thirteenth pose of Leupeptin on GLY 92 and TYR93 residues with H bond of falcipain-III protein 3BPM PDB; and

FIG. 11 illustrates 90I in white and Leupeptin in red using various poses.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

Primary Approach

Strategy I. Antimalarial Drug Using Novel Molecule

FIG. 2A illustrates a molecular structure of an anti-pathogenic compound, known as 90I (diethylene glycol dibenzoate), according to an exemplary embodiment of the present general inventive concept.

FIG. 2B illustrates a molecular structure of the anti-pathogenic compound, known as 90I, according to an exemplary embodiment of the present general inventive concept.

The first method will test use of a highly active antireroviral drug as an antimalarial drug. The highly active antiretroviral drug is based on a novel molecule that has been discovered within an herb located in Ethiopia that may be used to treat subjects (e.g., people, animals, etc.) infected with a pathogen, which in this case is malaria. However, although treatment is the term used, treatment may also include prophylaxis (i.e. prevention) and/or vaccination, such that the treatment may inhibit replication of and/or kill the pathogen. The observed window of efficacy at a traditional herbal treatment center in Ethiopia, used in treating AIDS patients, served as the basis for undertaking the developmental investigation of the crude product for further drug development investigation.

Furthermore, although the pathogen is identified as malaria, 90I may be used to treat any pathogen including a virus, bacteria, protozoan (i.e. parasite), and/or fungal.

Bacterial pathogens may include Mycobacterium tuberculosis Tuberculosis, Bacillus anthracis Anthrax, and Staphylococcus Sepsis aureus, but is not limited thereto.

Viral pathogens may include Adenoviridae, Mastadenovirus, Infectious canine hepatitis, Arenaviridae, Arenavirus, Lymphocytic choriomeningitis, Caliciviridae, Norovirus, Norwalk virus infection, Coronaviridae, Coronavirus, Severe Acute Respiratory Syndrome, SARS-CoV, SARS-CoV-2, Torovirus, Filoviridae, Marburgvirus, Viral hemorrhagic fevers, Ebolavirus, Viral hemorrhagic fevers, Flaviviridae, Flavivirus, West Nile Encephalitis, Hepacivirus, Hepatitis C virus infection, Pestivirus, Bovine Virus Diarrhea, Classical swine fever, Hepadnaviridae, Orthohepadnavirus, Hepatitis, Herpesviridae, Simplexvirus, cold sores, genital herpes, bovine mammillitis, Varicellovirus, chickenpox, shingles, abortion in horses, encephalitis in cattle, Cytomegalovirus, infectious mononucleosis, Mardivirus, Marek's disease, Orthomyxoviridae, Influenzavirus A, Influenza, Influenzavirus B, Influenza, Papillomaviridae, Papillomavirus, Skin warts, skin cancer, cervical cancer, Picornaviridae, Enterovirus, Polio, Rhinovirus, Common cold; Aphthovirus, Foot-and-mouth disease, Hepatovirus, Hepatitis, Poxviridae, Orthopoxvirus, Cowpox, vaccinia, smallpox, Reoviridae, Rotaviruses, Diarrhea, Orbivirus, Blue tongue disease, Retroviridae Gammaretrovirus, Feline leukemia, Deltaretrovirus, Bovine leukemia, Lentivirus, Human immunodeficiency, FIV, and SIV, Rhabdoviridae, Lyssavirus, Rabies, Ephemerovirus, Bovine ephemeral fever, Togaviridae, Alphavirus, and Eastern and Western equine encephalitis, but is not limited thereto.

Parasitic pathogens may include Plasmodium, Malaria, Leishmania, and Leishmaniasis, but is not limited thereto.

Fungal pathogens may include Aspergillis, Candida, Coccidia, Cryptococci, Geotricha, Histoplasma, Microsporidia, and Pneumocystis, but is not limited thereto.

As such, 90I may also be an anti-pathogenic compound that is applicable to different diseases and/or infections.

The Ethiopian region may be characterized by a wide range of ecological, edaphic, and climatic conditions that account for the wide diversity of its biological resources, both in terms of flora and faunal wealth. The plant genetic resources of the country exhibit an enormous diversity as seen in the fact that Ethiopia is one of the twelve Vavilov Centers of origin for domesticated crops and their wild and weedy relatives. According to recent studies, it is estimated that there are more than seven thousand species of flowering plants recorded in Ethiopia, of which at least twelve percent are probably endemic.

Medicinal plants may comprise one of the important components of Ethiopian vegetation. On record, there may be six hundred species of medicinal plants constituting a little over ten percent of Ethiopia's vascular flora. The medicinal plants may be distributed all over the country, with greater concentration in the south and southwestern parts of the country. Woodlands of Ethiopia may be the source of most of the medicinal plants, followed by the montane grassland and/or dry montane forest complex of the plateau. Other important vegetation types for medicinal plants may be the evergreen bushland and rocky areas.

As such, an herbal extract may be extracted from the herb from Ethiopia. The herbal extract may include a glycol derivative. Moreover, the glycol derivative may include diethylene glycol dibenzoate. An anti-pathogenic compound may include diethylene glycol dibenzoate to treat human immunodeficiency virus (HIV and/or HIV-1).

HIV-1 may have the best complicated strategy for survival than any microbial known to man. HIV-1 may replicate more than one billion virion every day in every patient, but its replication process is error prone when the reverse transcriptase (RT) (i.e. an enzyme that catalyzes the formation of DNA from an RNA template in reverse transcription) transcribes the viral RNA to DNA.

HIV-1 in vitro data analysis of 90I, via Bioassay Guided Fractionation, may confirm observed in vivo data and was used to isolate a lead molecule.

Full blown in vitro studies may demonstrate astonishing results that surpass any known commercially available antiretroviral (ARV) and/or highly active antiretroviral therapy (HAART) drugs currently on the market.

Referring to FIGS. 2A and 2B, an anti-pathogenic agent and/or the anti-pathogenic compound may be identified as 90I (or 90i). The anti-pathogenic compound may be derived from the herbal extract identified as H2K1001. H2K1001 and/or 90I may be a natural product that was isolated using the Bioassay Guided Fractionation, and further purified, molecularly characterized (i.e. characterizing at the molecular level without any effect of environment or development or physiological state of the organism), and not only found to be highly potent against all HIV-1 strains, but also immunogenic with unique multiple modes of action (i.e. functional or anatomical change at a cellular level, resulting from exposure of a living organism to a substance), potently effective against both reverse transcriptase and a protease (PR) enzyme (i.e. an enzyme which breaks down proteins and peptides). The essence of combination drug therapy, HAART regiments, may be its effectiveness against all HIV-1 strains that is potent enough to bring the viral load down to an undetectable level. This may be achieved by combining an RT and a PR combination synergy to affect multiple modes of action.

Referring again to FIGS. 2A and 2B, the molecular structure and molecular modeling study of H2K1001 and/or 90I may exhibit a number of properties that synergize with the outcome of the cell-based mode of action studies, early and/or late intervention. From a geometric angle, 90I may have a stable tetrameric structure like other antiretroviral compounds. From topographic 3D view, 90I has a “butterfly” moiety (i.e. a part of a molecule which is typically given a name as it can be found with other kinds of molecules as well) resembling that of the reverse transcriptase. From an angle of symmetry, 90I has a perfect vertical symmetry that divides the molecule in a mirror image isosteres (i.e. molecules or ions with similar shapes and often electronic properties) like the protease enzyme.

Additionally, 90I may be a bipolar molecule with a central domain flanked by aromatic rings (i.e. property of cylic (ring-shaped), planar structures with a ring of resonance bonds that give increased stability compared to other geometric or connective arrangements with the same set of atoms, such that the molecule has low reactivity with other substances) at both ends, as a dimer (i.e. an oligomer (a polymer whose molecules consist of relatively few repeating units) composed of two monomers that are similar in structure and jointed by a chemical bond) formation of the protease enzyme. Molecularly, 90I's central domain may be heavily enriched with molecular oxygen and hydrogen with an extreme binding affinity (i.e. strength of the binding interaction between a single biomolecule (e.g., a protein or DNA) to its ligand/binding partner (e.g., drug or inhibitor)). The flanking aromatic rings may be in a state of dynamic pi electron (i.e. an electron which resides in the pi bond(s) of a double bond or a triple bond, or in a conjugated p orbital) resonance.

Furthermore, 90I may be a molecule with multiple charges and high affinity at various levels of chemical bonding, hydrogen bonding, covalent bonding, ionic bonding and van der wall bonding (i.e. Van der Waals force, which describes attraction and repulsions between atoms, molecules, and surfaces, as well as other intermolecular forces that differ from covalent and ionic bonding in that they are caused by correlations in the fluctuating polarizations of nearby particles).

Also, 90I may include a flexible polymerizing molecule that could easily form a dimer, a tetramer (i.e. a polymer comprising four monomer units), and a polymer that makes a multimeric web like networking. A highly active inter-penetrating polymeric nature of this molecule may provide it with unique property of rearranging the position of the binding residues, repositioning of the interacting residues, intercalating between molecules, super-imposing over a molecule, flexing in active site cleft and pockets, forming bridges between molecules, oligomerizing with the interacting molecule, causing axis rotation, out-reaching sub-units in unreachable pockets, protruding in solvent channels, and creating channels.

The initial evaluation of 90I in the CEM-SS (i.e. human T-cells that are permissive to HIV-1 infection) cell line demonstrated significant antiviral activity against the RF laboratory strain of HIV-1. The purified fraction 90I may yield a fifty percent effective concentration (EC50) of 0.020 ug/ml. At the same time, there was no discernible drug induced toxicity in this study (50% inhibitory concentration (1050) was greater than the high-test concentration of 100 ug/ml). The resulting therapeutic index (T1=IC50/EC50) of greater than 5000 suggests a highly active compound.

In vitro data also revealed miraculous increased production of gamma interferon (i.e. a dimerized soluble cytokine that is the only member of the type II class of interferons and is a product of human leukocytes and human lymphocytes), which may be the result of Th1 subset (i.e. a subset of T lymphocytes that express CD4 and are known as T helper cells, they produce cytokines, specifically Th1-type cytokines) boosting. Moreover, the increase in gamma interferon may have a far greater impact on reversing the course of HIV-1 infection. Furthermore, 90I may be highly active in restoring immune system competence. Therefore, further evaluation of 90I in malaria cell-lines may provide the opportunity to find new clinical uses for an established HIV antiprotease patented drug. Also, the purpose of this approach is to lower the costs and shorten the timeframe of product development.

As previously mentioned, this drug (i.e. 90I and/or H2K1001) was developed from an Ethiopian Herb. In comparison to other commercial retrovirus drugs, 90I may include several unique properties, such as working as both reverse transcriptase and/or protease enzyme, against sensitive and resistant strains as well as boosting the immune surveillance system. More specifically, 90I may demonstrate high effectiveness against the protease enzyme that makes it highly attractive as a novel antimalarial drug against malaria.

FIG. 3 illustrates a molecular structure of chloroquine.

As discussed above, a potential new target for antimalarial chemotherapy includes the malarial proteases that are required for erythrocytic invasion and degradation of hemoglobin by parasites for its survival. 90I will be tested in vitro against sensitive and resistant P. falciparum using chloroquine as a control drug to determine median tissue culture infectious dose (TCID50) (i.e. a method used when verifying viral titer that signifies the concentration at which fifty percent of the cells are infected when a test tube or well plate upon which cells have been cultured is inoculated with a diluted solution of viral fluid), lethal dose fifty percent (TLD50), and therapeutic index 50 (TI50) (i.e. a ratio that compares the blood concentration at which a drug becomes toxic and the concentration at which the drug is effective). In other words, a combination drug assay between 90I and chloroquine will be set to determine percent synergy and/or antagonism using both sensitive and/or resistant strains.

A chemometric (i.e. the use of mathematical and statistical methods to improve the understanding of chemical information and to correlate quality parameters or physical properties to analytical instrument data) quantitative structure-activity relationship (QSAR) study (i.e. a technique that tries to predict the activity, reactivity, and properties of an unknown set of molecules based on analysis of an equation connecting the structures of molecules to their respective measured activity and property) was performed on 93 ahalcones derived from structure II against a strain of chloroquine-resistant human malarial parasite, P, falciparum, equivalent to W2 strain. The 90I geometric configuration may exhibit similar crosstalk (i.e. one or more components of one signal transduction pathway affects another).

Alternative Approaches a Follows:

Strategy II. Antimalarial Drug Development from five Ethiopian Plants

The second method of antimalarial treatment may use a plurality of secondary Ethiopian plants. The plurality of second Ethiopian plants may include a Withania Somnifera leaf and/or root, a Eucelia root aqueous extract, Phytolacca dodecandra, Vernonia galamensis ethiopica, and Moringa, but is not limited thereto.

In vivo testing of the Withania Somnifera leaf and/or root and the Eucelea root aqueous extract showed promising results against malaria in a previous study. Moreover, the Phytolacca dodecandra, the Vernonia galamensis ethiopica (i.e. a natural epoxy vernolic acid), and the Moringa may be included based on ethnomedical history and the field research prioritization serves as the initial biological screen. Phytochemical screening will be initiated using two types of solvents, polar and non-polar, at a first stage. Polar solvents are anticipated to provide the presence of tannins, polysaccharides, and proteins in the aqueous extracts, whereas non-polar solvent extracts may provide the presence of saponins, tannins, flavonoids, and terpenoids in methanol. Moreover, the phytochemical screening will be tested in vitro to also determine TCID50, TLD50, and TI50

Strategy Ill. Antimosquto Bed Net & Repellent from Ethiopian Plant

The third method is to develop a strong mosquito repellant insecticide extracted from the Vernonia galamensis ethiopica and the Phytolacca dodecandra. More specifically, a unique epoxy molecule from the Vernonia galamensis ethiopica has been found to have multiple industrial applications including plasticizers, as well as a stabilizer for plastic formulation, coating material, perfume, medicine, lubricant for mechanical components, an ingredient for ink formulations, pesticide, insect repellant, and/or pharmaceutical applications. Furthermore, Phytolacca dodecandra are effective for molluscicidal, larvicidal, fungicidal, and spermacidal activities (Lemma, A. Et al., 1979).

Also, mosquito larvae are susceptible to Phytolacca dodecandra to lethal effect. As such, a broad insecticide mosquito net will be prepared from a combination of the indigenous plant polymers in accordance with WHO/CDC/CPE/Whopes/99.4 regulations.

The fundamental underlying advantage that 90I and other molecules extracted from the plurality of secondary Ethiopian plants, identified above, have in comparison to the current treatments used for malaria may include multiple molecular modes of action that parallels to not one, but all currently used treatments (i.e. antimalarial, protease inhibitors, and interferon producer), multiple natural lead isolates identified (i.e. at least six total lead molecules identified as effective against malaria), multiple modes of application (i.e. drug formulation and development of repellent through use of combined six lead molecules including 90I), highly active (HAART), proven effective against resistance, such that promoting use of this drug without the need of combinatorial drugs being required, a natural product, provides a boost to the immune system, reverse latent infection, highly effective in brain cells, non-toxic, and affordable, but is not limited thereto.

Below is an in-silico analysis of 90I when comparing it to other control drugs that are currently used as antimalaria inhibitors and/or have been known to inhibit P. falciparum in vitro, namely chloroquine, hydroxychloroquine, Remdisivir, and Lopinavir/Ritonavir (LPV/RTV) combination with chloroquine, trans-Epoxysuccinyl-L-leucylamido(4-guanidino)butane (E64) (i.e. a protease inhibitor), and Leupeptin (i.e. a natural protease inhibitor).

In vitro results of 90I may yield a high Therapeutic Index, excellent EC50, and 1050 against HIV, as discussed above. Moreover, a toxicity test may be performed for safety assessment issues and risks for humans, identified through hepatotoxicity (i.e. chemical-driven liver damage), genotoxicity (i.e. chemical information that damages the genetic information within a cell causing mutations), immunogenicity (i.e. ability of a foreign substance, such as an antigen, to provoke an immune response in the body of a human or other animal), general toxicology, nephrotoxicity (i.e. renal toxicity occurs when a drug or toxin damages the kidneys), and secondary drug metabolite pharmacology studies.

A toxicity protocol may be utilized to assess antimalaria drug safety, the toxicity protocol was already designed and established for HIV and the other infectious diseases under investigation. Also, many of the issues previously noted for current malaria therapies will be circumvented by thoroughly evaluating the 90I compound in response to contact with a biological system. Previous therapies failed to analyze the biological responses, such as inhibition of key enzymes, interaction with receptors, interaction with other therapies patients received, etc.

Each of the current therapies noted above, chloroquine, hydroxychloroquine, Remdisivir, LPV/RTV, E64, and Leupeptin (See https://www.rcsb.org/3d-view/3BPM/1) were used in parallel comparison analysis utilizing virtual screening based on prediction.

1. Comparison of 90I to chloroquine.

Although the molecular mechanism by which chloroquine exerts its effects on the malarial parasite Plasmodium falciparum remains unclear, the drug has previously been found to interact specifically with the glycolytic enzyme lactate dehydrogenase from the parasite. In this study we have determined the crystal structure of the complex between chloroquine and P. falciparum lactate dehydrogenase. The bound chloroquine is clearly seen within the NADH binding pocket of the enzyme, occupying a position similar to that of the adenyl ring of the cofactor. Chloroquine hence competes with NADH for binding to the enzyme, acting as a competitive inhibitor for this critical glycolytic enzyme. Specific interactions between the drug and amino acids unique to the malarial form of the enzyme suggest this binding is selective. Inhibition studies confirm that chloroquine acts as a weak inhibitor of lactate dehydrogenase, with mild selectivity for the parasite enzyme. As chloroquine has been shown to accumulate to millimolar concentrations within the food vacuole in the gut of the parasite, even low levels of inhibition may contribute to the biological efficacy of the drug. The structure of this enzyme-inhibitor complex provides a template from which the quinoline moiety might be modified to develop more efficient inhibitors of the enzyme. (See https://www.ncbi.nlm.nih.gov/pubmed/10187806).

The following excerpt is from “In-Silico Assessment of Various PDB Entries of PfLDH Enzyme for their Use in SBDD.” (See https://cheminformatics.imedpub.com/insilico-assessment-of-various-pdb-entries-ofpfldh-enzyme-for-their-use-in-sbdd.php?aid=9812).

In this case, the objective was to perform in silico structural assessment analysis of protein database entries of Plasmodium falciparum lactate dehydrogenase (PfLDH) enzyme, an important target for designing of anti-malarial drugs.

Plasmodium falciparum LDH (PfLDH) is a 316 amino acid protein, coded by a single gene on chromosome 13, and is expressed as a 1.6-kb mRNA. The amino add sequence predicted from genomic and cDNA sequencing indicates that essential catalytic residues, such as His195, Asp168, Arg109 and Arg171 are crucial for its activity. Asp168 and His195 act as hydrogen donors; side chain of Arg171 interacts with the carboxylate of pyruvate; side chain of Arg109 interacts with the ketone oxygen of pyruvate leading to polarization of the ketone carboxyl and hydride attack from NAD; proline is critical active site residue which defines substrate and cofactor binding sites. Asn197, Lys102, and Leu163 define the conserved active site.

The following information is from the Protein Data Bank (PDB), ID: 10ET, Chloroquine binds in the cofactor binding site of PfLDH. (See https://www.rcsb.org/3d-view/10ET/1).

The following excerpt is from “Chloroquine Binds in the Cofactor Binding Site of Plasmodium falciparum Lactate Dehydrogenase.” (See https://www.jbc.org/content/274/15/10213.full).

A second protein from PDB that can be used is 10EQ. (See https://www.rcsb.org/structure/10EQ).

Below we show an in-silico result of chloroquine and hydroxychloroquine and a PDB 10EQ protein with identified Lactate Dehydrogenase.

Although, 90I interacts with residues and/or a binding domain of a substrate to interrupt the protein from functioning, it also interacts allosterically (i.e. of, relating to, undergoing, or being a change in the shape and activity of a protein (such as an enzyme) that results from combination with another substance at a point other than the chemically active site) to affect the protein's active site without actually interacting with the binding domain and the active site.

FIG. 4 illustrates a first pose of 90I including two hydrogen bonds (H bonds), according to an exemplary embodiment of the present general inventive concept.

FIG. 5 illustrates a first pose of chloroquine including one H bond.

Referring to FIG. 4, 90I may form two hydrogen bonds.

Referring to FIG. 5, conversely, chloroquine forms only one H bond in an interaction with a cavity pocket where THR97 and GLY29 are located. There is a cavity and pocket at these residues.

FIG. 6 illustrates the first pose of chloroquine disposed in a pocket.

Referring to FIG. 6, chloroquine shows high affinity and a short H bond distance.

FIG. 7A illustrates the first pose of 90I disposed in the pocket, according to an exemplary embodiment of the present general inventive concept.

Referring to FIG. 7A, 90I shows high affinity and a short H bond distance.

FIG. 7B illustrates a second pose of 90I in the pocket, according to an exemplary embodiment of the present general inventive concept.

Referring to FIG. 7B, 90I again shows high affinity and a short H bond distance. Conversely, a second pose of chloroquine cannot achieve this result.

FIG. 7C illustrates another view of the first pose of 90I in the pocket, according to an exemplary embodiment of the present general inventive concept.

FIG. 7D illustrates another view of the second pose of 90I in the pocket, according to an exemplary embodiment of the present general inventive concept.

FIG. 7E illustrates a third pose of 90I in the pocket, according to an exemplary embodiment of the present general inventive concept.

FIG. 7F illustrates a fourth pose of 90I in the pocket, according to an exemplary embodiment of the present general inventive concept.

FIG. 7G illustrates a fifth pose of 90I in the pocket, according to an exemplary embodiment of the present general inventive concept.

FIG. 7H illustrates a sixth pose of 90I in the pocket, according to an exemplary embodiment of the present general inventive concept.

FIG. 7I illustrates a first surface view of the first pose of 90I in the pocket, according to an exemplary embodiment of the present general inventive concept.

FIG. 7J illustrates a second surface view at a different angle with respect to the first surface view of the first pose of 90I in the pocket including two hydrogen bonds in dotted yellow lines, according to an exemplary embodiment of the present general inventive concept.

FIG. 7K illustrates 90I disposed at a deeper portion of the pocket including the two hydrogen bonds in dotted yellow lines, according to an exemplary embodiment of the present general inventive concept.

The scoring we see below and care about the most, per autodock vina, is the first pose, and the second best score also can be considered if it generates a good H bond distance root-mean-square deviation (RMSD) value. Corresponding to the best short distance, we then look for affinity. The less the kcal/mol the better since it means it is consuming the least energy to bind.

TABLE 1

| | affinity | dist from best mode | |
|---|---|---|---|
| mode | (kcal/mol) | rmsd l.b. | rmsd u.b. |
| 1 | −6.9 | 0.000 | 0.000 |
| 2 | −6.8 | 8.498 | 8.083 |
| 3 | −6.8 | 22.225 | 24.752 |
| 4 | −6.6 | 21.350 | 23.682 |
| 5 | −6.6 | 21.517 | 24.688 |
| 6 | −6.5 | 5.949 | 10.360 |
| 7 | −6.2 | 5.926 | 11.047 |
| 8 | −6.1 | 22.158 | 24.198 |
| 9 | −6.1 | 27.948 | 29.170 |
| 10 | −6.1 | 12.619 | 16.075 |
| 11 | −6.0 | 12.579 | 15.518 |
| 12 | −6.0 | 28.358 | 29.805 |
| 13 | −5.9 | 24.126 | 28.417 |
| 14 | −5.8 | 25.671 | 29.884 |
| 15 | −5.7 | 26.898 | 30.900 |
| 16 | −5.7 | 14.512 | 17.570 |
| 17 | −5.7 | 19.763 | 21.829 |
| 18 | −5.7 | 5.893 | 8.247 |
| 19 | −5.6 | 6.889 | 11.258 |
| 20 | −5.6 | 26.648 | 29.949 |

Writing output . . . done.

Referring to Table 1, a log file for 90I with different affinity values is included.

The first pose and the second pose may provide excellent distance between 90I molecule and residues of the protein.

Zero RMSD is as close as atoms can get to each other. So, 0.49 Angstrom (A) is very close. A value of less than 3 A is acceptable.

According to previous studies found in peer-reviewed literature, there may be more significance of binding with a protein-ligand complex and having the lowest energy, such that there is a better binding affinity. Moreover, a benchmark being 5 kcal/mol or less is better.

When a molecule is subjected to dock at a receptors cavity according to a geometrical and/or a physical nature of a selected cavity, user defined numbers of the most complimentary and highest binding affinity having conformations of the ligand are generated. Subsequently, they were aligned to compare their displacement and conformational changes and the results are reported in RMSD. The highest binding affinity having conformation will be compared to itself, so it will be always zero A. The second highest binding affinity having conformation will be compared to the highest. The third highest binding affinity having conformation will be compared to the second highest binding affinity, and additional results may follow a similar pattern.

All RMSD values may be calculated relative to the best mode (i.e. the first model and/or result) and use only movable heavy atoms (i.e. only ligand atoms, not hydrogen). An RMSD upper bound may match each atom in one conformation with itself in another conformation, thereby ignoring any symmetry. An RMSD lower bound may match each atom in one conformation with the closest atom of the same element type in the another conformation. Since this method is based on atom type and distance, it may no longer be a one-to-one comparison. For example, it might use eight atoms from conformer A and ten atoms from conformer B to calculate RMSD. The RMSD lower bound may return the highest RMSD out of RMSD(A, B) and RMSD(B, A). (See https://www.researchgate.net/post/How_to_analyze_Autodock_vina_results_How_to_int erpret_Autodock_vina_score_values_What_is_basic_cutoff parameters_for_Autodock_vina_results).

As mentioned previously, a binding with a protein-ligand complex and having the lowest energy, results in a better binding affinity. The benchmark is 5 kcal/mol or less is better, and an H bond of less than 3 Argon RMSD to be an ideal distance from the residue atom that interacts to create a hydrogen bond.

TABLE 2

| | affinity | dist from best mode | |
|---|---|---|---|
| mode | (kcal/mol) | rmsd l.b. | rmsd u.b. |
| 1 | −6.7 | 0.000 | 0.000 |
| 2 | −6.7 | 24.621 | 27.106 |
| 3 | −6.6 | 3.314 | 7.859 |
| 4 | −6.5 | 2.108 | 3.056 |
| 5 | −6.1 | 4.097 | 7.604 |
| 6 | −6.1 | 26.040 | 27.534 |
| 7 | −6.1 | 1.546 | 2.070 |
| 8 | −6.0 | 8.860 | 11.383 |
| 9 | −6.0 | 2.161 | 5.602 |
| 0 | −5.9 | 9.172 | 11.755 |
| 11 | −5.9 | 25.771 | 27.280 |
| 12 | −5.9 | 23.135 | 25.986 |
| 13 | −5.9 | 3.022 | 6.854 |
| 14 | −5.8 | 41.223 | 42.357 |
| 15 | −5.8 | 5.651 | 8.790 |
| 16 | −5.7 | 9.882 | 13.167 |
| 17 | −5.7 | 3.954 | 7.735 |
| 18 | −5.7 | 8.001 | 10.890 |
| 19 | −5.7 | 1.923 | 2.939 |
| 20 | −5.6 | 8.259 | 11.629 |

Writing output . . . done.

Referring to Table 2, a log file for chloroquine with different affinity values is included.

The second pose of chloroquine has very high distance of H bond, approximately 25 A RMSD, which is less favorable than that of 90I at 0.4 A RMSD value. In other words, these values illustrate the distance between 90I and the residue (i.e. the atom being interacted with) compared to the distance between chloroquine and the residue.

TABLE 3

| | affinity | dist from best mode | |
|---|---|---|---|
| mode | (kcal/mol) | rmsd l.b. | rmsd u.b. |
| 1 | −6.7 | 0.000 | 0.000 |
| 2 | −6.7 | 24.621 | 27.106 |
| 3 | −6.6 | 3.314 | 7.069 |
| 4 | −6.5 | 2.108 | 3.956 |
| 5 | −6.1 | 4.097 | 7.604 |
| 6 | −6.1 | 26.940 | 27.534 |
| 7 | −6.1 | 1.546 | 2.070 |
| 8 | −6.0 | 8.860 | 11.383 |
| 9 | −6.0 | 2.161 | 5.602 |
| 10 | −5.9 | 9.172 | 11.755 |
| 11 | −5.9 | 25.771 | 27.280 |
| 12 | −5.9 | 23.135 | 25.986 |
| 13 | −5.9 | 3.022 | 6.854 |
| 14 | −5.8 | 41.223 | 42.357 |
| 15 | −5.8 | 5.651 | 8.790 |
| 16 | −5.7 | 9.882 | 13.167 |
| 17 | −5.7 | 3.954 | 7.735 |
| 18 | −5.7 | 8.001 | 10.890 |
| 19 | −5.7 | 1.923 | 2.939 |
| 20 | −5.6 | 8.259 | 11.629 |

Writing output . . . done.

Referring to Table 3, log file for chloroquine with different affinity values is included.

Referring again to Table 3, the second pose of chloroquine shows long distance between atoms compared to the second pose of 90I, as described above. More specifically, the second pose of chloroquine shows 25 A, but the second pose of 90I shows 0.4 A, which is a short distance H bond.

TABLE 4

| | affinity | dist from best mode | |
|---|---|---|---|
| mode | (kcal/mol) | rmsd l.b. | rmsd u.b. |
| 1 | −6.9 | 0.000 | 0.000 |
| 2 | −6.7 | 24.209 | 26.691 |
| 3 | −6.4 | 4.486 | 7.573 |
| 4 | −6.0 | 1.311 | 2.022 |
| 5 | −6.0 | 8.445 | 11.000 |
| 6 | −6.0 | 4.792 | 6.787 |
| 7 | −5.9 | 2.761 | 5.862 |
| 8 | −5.9 | 24.590 | 26.923 |
| 9 | −5.9 | 1.848 | 2.704 |
| 10 | −5.9 | 4.841 | 7.874 |
| 11 | −5.8 | 24.086 | 26.482 |
| 12 | −5.8 | 19.226 | 20.550 |
| 13 | −5.8 | 9.050 | 11.734 |
| 14 | −5.7 | 7.992 | 10.629 |
| 15 | −5.7 | 14.591 | 17.163 |
| 16 | −5.7 | 2.395 | 3.458 |
| 17 | −5.7 | 2.945 | 6.205 |
| 18 | −5.7 | 9.281 | 11.817 |
| 19 | −5.6 | 2.291 | 2.721 |
| 20 | −5.6 | 25.581 | 27.078 |

Writing output . . . done.

Referring to Table 4, a log file for hydroxychloroquine with different affinity values is included.

Referring again to Table 4, hydroxychloroquine performs similarly to chloroquine. Moreover, hydroxychloroquine is less toxic than chloroquine. However, 90I may have the least toxicity as per HIV in vitro tests. Also, the second pose for 90I shows shorter distance than a second pose of hydroxychloroquine on this log.

2. Leupeptin

Leupeptin (i.e. N-acetyl-L-leucyl-L-leucy-L-argininal) is a naturally occurring protease inhibitor that can inhibit cysteine, serine, and threonine peptidases. It is often used during in vitro experiments when a specific enzymatic reaction is being studied (See https://www.rcsb.org/3d-view/3BPM/1).

A plurality of residues of a falcipain-III protein interact with leupeptin on the following residues: Gln45, Gly49, Ser50, Cys51, Trp52, Tyr90, Gly91, Gly92, Tyr93, Asp164, Glu176, Asn182, His183 (See https://pdfs.semanticscholar.org/7207/ed990d2e93cf74f40dc9a6ba7bfb4b5fbe35.pdf).

Leupeptin, from PDB protein was used (See https://www.rcsb.org/3d-view/3BPM/1).

In summary, treatment of cultures with the irreversible broad-spectrum cysteine protease inhibitor E64, which does not affect serine proteases, had multiple effects on P. falciparum culture including: 1) blocked parasite release, acting within the last minutes of the cycle, 2) induced formation of parasite clusters, limited by a single erythrocyte membrane, and 3) interfered with hemoglobin degradation in food vacuoles during prolonged treatment.

All three of these effects were also seen with protease inhibitors of various specificities, including leupeptin and calpeptin (Aoyagi et al., 1969; Tsujinaka et al., 1988) at concentrations 10 μg/ml and 1 μM, respectively. (See https://www.ncbi.nlm.nih.gov/pmc/articles/PMC2883916/).

See https://www.ncbi.nlm.nih.gov/pubmed/23995310. Also, https://www.ncbi.nlm.nih.gov/pmc/articles/PMC3317066/).

Methods

The proteins, falcipain-II and falcipain-III, together with leupeptin were retrieved from the PDB, and subsequently, leupeptin was compared with 90I. 90I was screened via docking simulation at the active site cavity of falcipain-II and falcipain-III by using AutoDock and Autodock vina.

Please see below in-silico result for comparison of 90I with leupeptin.

FIG. 8A illustrates Leupeptin in blue and F3 P. Falciparum in gray from Protein Data Bank (PDB), including one section of F3 having residues as green dots.

Referring to FIG. 8A, the image shows an x-ray that has been crystalized. Also, the H bond cavity pocket is not shown.

First, a crystal structure of Falcipain-3 with its inhibitor, Leupeptin (see https://www.rcsb.org/3d-view/3BPM/1) was obtained, then pymol was used to identify the residues, which are colored as shown above. Additionally, Autodock was used to identify the same residues and run the process for docking.

Falcipain-2 and falcipain-3 are critical hernoglobinases of Plasmodium falciparum, the most virulent human malaria parasite. It has been determined that the 2.9 A crystal structure of falcipain-2 in complex (i.e. a substance, either an ion or an electrically neutral molecule, formed by the union of simpler substances (as compounds or ions) and held together by forces that are chemical (i.e. dependent on specific properties of particular atomic structures) rather than physical) with the epoxysuccinate E64 and the 2.5 A crystal structure of falcipain-3 in complex with the aldehyde leupeptin. These complexes represent the first crystal structures of plasmodial cysteine proteases with small molecule inhibitors and the first reported crystal structure of falcipain-3.

Structural analyses indicate that the relative shape and flexibility of the S2 pocket are affected by a number of discrete amino acid substitutions. The cumulative effect of subtle differences, including those at “gatekeeper” positions, may explain the observed kinetic differences between these two closely related enzymes.

FIG. 8B illustrates Leupeptin in green and F3 P. Falciparum in gray from PDB.

Referring to FIG. 8B, the Leupeptin from FIG. 8A was removed and replaced with a ligand obtained from Pubchem (see https://pubchem.ncbi.nlm.nih.gov/compound/Leupeptin), then configured to run with autodock and the result is shown in green interacting similarly as in FIG. 8A. Similar to FIG. 8A, the x-ray was crystalized, which confirms in-silico is correct when compared to a real x-ray.

FIG. 8C illustrates a first pose of Leupeptin in green interacting with ASN 182 and F3 P. Falciparum in gray including residues in blue.

FIG. 9A illustrates the first pose of 90I using residues of falcipain-III protein 3BPM PDB, according to an exemplary embodiment of the present general inventive concept.

FIG. 9B illustrates the fifth pose of 90I on residues of falcipain-III protein 3BPM PDB, according to an exemplary embodiment of the present general inventive concept.

Referring to FIG. 9B, the fifth pose of 90I shows −6.3 kcal per mol and 2.375 A distance H bond RMSD.

FIG. 9C illustrates the first pose of 90I on residues of falcipain-III protein 3BPM PDB, according to an exemplary embodiment of the present general inventive concept.

Referring to FIG. 9C, the first pose of 90I shows −6.0 kcal per mol and a 12.467 A distance H bond RMSD showing strong 3 H bonds.

FIG. 9D illustrates a twelfth pose of 90I on Gly 49 residues of falcipain-III protein 3BPM PDB, according to an exemplary embodiment of the present general inventive concept.

Referring to FIG. 9D, the twelfth pose of 90I shows −6.0 kcal per mol and 9.562 A distance H bond RMSD.

FIG. 9E illustrates a fifteenth pose of 90I on GLN 45 and Gly 92 residues of falcipain-III protein 3BPM PDB, according to an exemplary embodiment of the present general inventive concept.

Referring to FIG. 9E, the fifteenth pose of 90I shows −5.9 kcal per mol and 5.180 A distance H bond RMSD.

FIG. 10A illustrates a second pose of Leupeptin on GLY 92 residues with a hydrogen bond of falcipain-III protein 3BPM PDB.

Referring to FIG. 10A, the second pose of Leupeptin shows −6.9 kcal per mol and 2.632 A distance H bond RMSD.

FIG. 10B illustrates a sixth pose of Leupeptin on GLY 92 and TYR93 residues with the hydrogen bond of falcipain-III protein 3BPM PDB.

Referring to FIG. 10B, the sixth pose of Leupeptin shows −6.8 kcal per mol and 2.302 A H bond distance RMSD.

FIG. 100 illustrates a seventh pose of Leupeptin on GLY 92 and TYR93 residues with the hydrogen bond of falcipain-III protein 3BPM PDB.

Referring to FIG. 100, the seventh pose of Leupeptin shows −6.8 kcal per mol and 2.302 A H bond distance RMSD.

FIG. 10D illustrates a thirteenth pose of Leupeptin on GLY 92 and TYR93 residues with H bond of falcipain-III protein 3BPM PDB.

Referring to FIG. 10D, the thirteenth pose of Leupeptin shows −6.6 kcal per mol and 2.332 H bond distance RMSD.

FIG. 11 illustrates 90I in white and Leupeptin in Red using various poses. Green indicates when 90i and Leupeptine are posing together showing similarities between the two.

Even when they are not together 90I is still interacting with all the residues mentioned separately showing that 90I is a stronger inhibitor with respect to Leupeptin.

3. Protease Inhibitors.

Plasmodium falciparum is one of the two protozoan parasites responsible for most of the world's cases of human malaria with the other being P. vivax. In 2010, there were an estimated 450 million clinical cases of P. falciparum malaria in the world. Based on where they live, an estimated 2.5 billion people were at possible risk of infection with P. falciparum as of 2005. Malaria remains one of the most important infectious disease problems in the world. The treatment and control of malaria are greatly limited by the increasing resistance of malaria parasites, particularly Plasmodium falciparum, to available drugs. New antimalarial agents, ideally directed against new targets, are therefore an urgent priority. Among the potential targets for drugs directed against P. falciparum are proteases that hydrolyze hemoglobin to provide amino acids for parasite protein synthesis. Multiple proteases appear to participate in this process, including the cysteine proteases falcipain-2 and falcipain-3. Inhibitors of these cysteine proteases block the hydrolysis of hemoglobin and thereby halt the development of cultured P. falciparum parasites. Efforts are therefore under way to discover inhibitors of falcipain-2/-3 with acceptable properties for new antimalarial drugs. (See https://www.ncbi.nlm.nih.gov/pmc/articles/PMC296233/).

A proprietary protease inhibitor drug based on 90I has been developed to test against P. falciparum.

The following excerpt is from “An in-silico test was used for Plasmodium falciparum Cysteine Protease Falcipain-2.” (See https://www.rcsb.org/structure/1yvb).

Among potential new targets for antimalarial chemotherapy are falcipain cysteine proteases. The best characterized of these proteases, falcipain-2 and falcipain-3, play key roles in the hydrolysis of hemoglobin by intraerythrocytic parasites. Inhibitors of falcipains demonstrate potent in vitro and in vivo antimalarial activity, and these proteases are the targets of efforts to develop novel cysteine protease inhibitors as new antimalarial drugs.

Results of structure-function studies were straightforward. As expected, the upstream portion of the falcipain-2 prodomain, which mediates protein trafficking, was not required for inhibitory activity. Indeed, only a small portion of the prodomain (Leu¹⁵⁵-Asp²¹⁶) was required for sub-nanomolar inhibition of the mature enzyme. We did not demonstrate inhibition by the isolated Leu¹⁵⁵-Asp²¹⁶ peptide, as production of this peptide proved difficult, but consideration of inhibition by a number of overlapping constructs clearly demonstrates that this peptide is sufficient for inhibition of falcipain-2. (See https://journals.plos.org/plosone/article?id=10.1371/journal.pone.0005694).

Since 90I is an HIV protease and reverse transcriptase inhibitor, it can also be used for malaria as a protease inhibitor. There has been at least one study conducted regarding the use of HIV protease inhibitors for malaria.

Aspartic proteases play key roles in the biology of malaria parasites and human immunodeficiency virus type 1 (HIV-1). We tested the activity of seven HIV-1 protease inhibitors against cultured Plasmodium falciparum. All compounds inhibited the development of parasites at pharmacologically relevant concentrations. The most potent compound, lopinavir, was active against parasites (50% inhibitory concentration [1050], 0.9 to 2.1 μM) at concentrations well below those achieved by ritonavir-boosted lopinavir therapy. Lopinavir also inhibited the P. falciparum aspartic protease plasmepsin II at a similar concentration (1050, 2.7 μM). These findings suggest that use of HIV-1 protease inhibitors may offer clinically relevant antimalarial activity. (See https://www.ncbi.nlm.nih.gov/pmc/articles/PMC1168637/).

90I may be a non-nucleoside reverse transcriptase inhibitor (NNRTI) and a protease inhibitor (PI).

Since 90I may be non-toxic and have high TI, it will be a good new drug candidate that will resolve the following problem described below.

The following is another excerpt: Our results support the intriguing possibility that HIV-infected individuals receiving protease inhibitor therapy may also benefit from an antimalarial effect due to inhibition of plasmepsins (i.e. a class of at least ten enzymes produced by P. falciparum). In vitro, lopinavir demonstrated potent activity against P. falciparum at concentrations well below those achievable with standard dosing of a ritonavir-boosted lopinavir regimen. Due to concerns regarding cost, toxicity, and potential selection of resistant viruses, it is unlikely that currently available HIV-1 protease inhibitors will gain roles as standard treatments for malaria. Nonetheless, it seems likely that, for select protease inhibitors, the concentrations achieved during chronic antiretroviral therapy will offer some protection against malaria. If standard regimens for HIV-1 offer chemoprophylaxis against malaria, particularly in children, in whom the burden of malaria is greatest, the clinical consequences of this effect will be great. However, it is unclear if in vitro results showing antimalarial activity of HIV-1 protease inhibitors predict clinical efficacy. Therefore, clinical trials to test the hypothesis that HIV-1 protease inhibitors confer protection against malaria are urgently needed. (See https://www.ncbi.nlm.nih.gov/pmc/articles/PMC1168637/).

Therefore, 90I and the proposed methods described above are all natural, low costing, and non-toxic treatments in targeting the most highly infectious parasitic disease which has crippled and burdened governments worldwide, especially third world countries. Moreover, 90I and the proposed methods may resolve the problems found in prior antimalarials. From the standpoint of a customer, most malaria cases include individuals who cannot afford the current available treatments. Investing in these proposed methods will alleviate the financial burden of the patients and decrease the need for treatment of side effects caused by the current available antimalaria drugs on the market.

### REFERENCES

The following reference(s) may provide exemplary procedural and/or other details supplementary to those set forth herein, and are specifically incorporated herein by reference.

Antimalarial natural products: a review, https://www.ncbi.nlm.nih.gov/pmc/articles/PMC4075661/.

Crystal Structure of Falcipain-3 with its inhibitor, Leupeptin, https://www.rcsb.org/3d-view/3BPM/1.

Chloroquine binds in the cofactor binding site of Plasmodium falciparum lactate dehydrogenase, https://www.ncbi.nlm.nih.gov/pubmed/10187806.

In-Silico Assessment of Various PDB Entries of PfLDH Enzyme for their Use in SBDD, https://cheminformatics.imedpub.com/insilico-assessment-of-various-pdb-entries-ofpfldh-enzyme-for-their-use-in-sbdd.php?aid=9812.

1CET, Chloroquine Binds in the Cofactor Binding Site of Plasmodium falciparum Lactate Dehydrogenase, https://www.rcsb.org/3d-view/10ET/1.

Chloroquine Binds in the Cofactor Binding Site of Plasmodium falciparum Lactate Dehydrogenase, https://www.jbc.org/content/274/15/10213.full.

1CEQ, Chloroquine Binds in the Cofactor Binding Site of Plasmodium falciparum Lactate Dehydrogenase, https://www.rcsb.org/structure/10EQ.

How to analyze Autodock vina results? How to interpret Audock vina score values? What is basic cutoff parameters for Autodock vina results?, https://www.researchgate.net/post/How_to_analyze_Autodock_vina_results_How_to_int erpret_Autodock_vina_score_values_What_is_basic_cutoff parameters_for_Autodock_vina_results.

Computation-based virtual screening for designing novel antimalarial drugs by targeting falcipain-III: A structure-based drug designing approach, https://pdfs.semanticscholar.org/7207/ed990d2e93cf74f40dc9a6ba7bfb4b5fbe35.pdf.

Irreversible effect of cysteine protease inhibitors on the release of malaria parasites from infected erythrocytes, https://www.ncbi.nlm.nih.gov/pmc/articles/PMC2883916/.

Structure-Function of Falcipains: Malarial Cysteine Proteases, https://www.ncbi.nlm.nih.gov/pmc/articles/PMC3317066/.

Leupeptin, https://pubchem.ncbi.nlm.nih.gov/compound/Leupeptin.

Antimalarial Activities of Novel Synthetic Cysteine Protease Inhibitors, https://www.ncbi.nlm.nih.gov/pmc/articles/PMC296233/.

1YVB, Plasmodium falciparum Cysteine Protease Falcipain-2, https://www.rcsb.org/structure/1yvb.

Regulatory Elements within the Prodomain of Falcipain-2, a Cysteine Protease of the Malaria Parasite Plasmodium falciparum, https://journals.plos.org/plosone/article?id=10.1371/journal.pone.0005694.

Antimalarial Activity of Human Immunodeficiency Virus Type 1 Protease Inhibitors, https://www.ncbi.nlm.nih.gov/pmc/articles/PMC1168637/.

The present general inventive concept may include a method for the treatment of malaria disease, including administering to a subject in need thereof of an anti-pathogenic compound, such that the anti-pathogenic compound is derived from an herbal extract.

The herbal extract may be a glycol derivative.

The glycol derivative may be diethylene glycol dibenzoate.

The malaria may be caused by Plasmodium falciparum.

The anti-pathogenic compound may boost an immune system of the subject.

Boosting the immune system may include revitalizing at least one of a dysfunctional monocyte and a dysfunctional macrophage.

Boosting the immune system may include stimulating production of gamma interferon.

Boosting the immune system may use only the anti-pathogenic compound.

The present general inventive concept may also include a method for the treatment of malaria disease, including administering to a subject in need thereof of an anti-pathogenic compound, such that the anti-pathogenic compound is derived from at least one Ethiopian plant.

The at least one Ethiopian plant may be at least one of Withania Somnifera leaf, Withania Somifera root, Eucelea root aqueous extract, Phytolacca dodecandra, Vernonia galamensis ethiopica, and Moringa.

The malaria may be caused by Plasmodium falciparum.

The present general inventive concept may also include a method of preventing at least one mosquito from crossing a barrier, including applying a mosquito repellant insecticide to the barrier, such that the mosquito repellent insecticide is derived from at least one Ethiopian plant.

The at least one Ethiopian plant may be at least one of Vernonia galamensis ethiopica and Phytolacca dodecandra.

The barrier may be a bed net.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A method for the treatment of malaria disease, comprising:
administering to a subject in need thereof of an anti-pathogenic compound,
such that the anti-pathogenic compound is derived from an herbal extract.

2. The method of claim 1, wherein the herbal extract is a glycol derivative.

3. The method of claim 2, wherein the glycol derivative is diethylene glycol dibenzoate.

4. The method of claim 1, wherein the malaria is caused by Plasmodium falciparum.

5. The method of claim 1, wherein the anti-pathogenic compound boosts an immune system of the subject.

6. The method of claim 5, wherein boosting the immune system includes revitalizing at least one of a dysfunctional monocyte and a dysfunctional macrophage.

7. The method of claim 5, wherein boosting the immune system includes stimulating production of gamma interferon.

8. The method of claim 5, wherein boosting the immune system uses only the anti-pathogenic compound.

9. A method for the treatment of malaria disease, comprising:
administering to a subject in need thereof of an anti-pathogenic compound,
such that the anti-pathogenic compound is derived from at least one Ethiopian plant.

10. The method of claim 9, wherein the at least one Ethiopian plant is at least one of Withania Somnifera leaf, Withania Somifera root, Eucelea root aqueous extract, Phytolacca dodecandra, Vernonia galamensis ethiopica, and Moringa.

11. The method of claim 9, wherein the malaria is caused by Plasmodium falciparum.

12. A method of preventing at least one mosquito from crossing a barrier, comprising:
applying a mosquito repellant insecticide to the barrier,
such that the mosquito repellant insecticide is derived from at least one Ethiopian plant.

13. The method of claim 12, wherein the at least one Ethiopian plant is at least one of Vernonia galamensis ethiopica and Phytolacca dodecandra.

14. The method of claim 12, wherein the barrier is a bed net.
